(19) United States
(12) Patent Application Publication
(10) Pub. No.: US 2006/0078516 A1
(43) Pub. Date: Apr. 13, 2006

(54) Sunless tanning system

(76) Inventors: Eliezer Parker, DN East Binyamin (IL); Philip Goodman, DN East Binyamin (IL)

Correspondence Address: SCHWEITZER CORNMAN GROSS & BONDELL LLP, 292 MADISON AVENUE - 19th FLOOR, NEW YORK, NY, 10017, US

(21) Appl. No.: 11/219,518

(22) Filed: Sep. 2, 2005

Related U.S. Application Data
(60) Provisional application No. 60/606,564, filed on Sep. 2, 2004.

(51) Int. Cl. A61K 9/70; A61K 8/00

(52) U.S. Cl. 424/59; 424/443

(57) ABSTRACT

A sunless tanning system comprising a mitt applicator and novel packaging therefor. The exterior of the mitt is impregnated with sunless tanning composition and the interior of the mitt is constructed to prevent the penetration of sunless tanning composition. The packaging is configured so that when it is opened there is a large aperture extending through the opening of the package to the interior of the mitt or such aperture can be readily formed while the mitt is in the packaging without touching the mitt. This allows the user to place the mitt on their hand and apply the sunless tanning composition without coming into contact with the sunless tanning composition.

### RELATED APPLICATION

This application claims the priority of provisional U.S. Application Ser. No. 60/606,564, filed Sep. 2, 2004.

### FIELD OF THE INVENTION

This invention relates generally to a personal sunless tanning system, and more particularly to a mitt-shaped applicator impregnated with sunless tanning composition and packaging therefor which allows a user to manually apply a sunless tanning composition in an efficient manner without inadvertently contacting the sunless tanning composition.

### BACKGROUND OF THE INVENTION

Sunless tanning compositions have become extremely popular because they can provide a user with a rich, natural looking tan in a short time period of time without health risks associated with traditional tanning methods involving exposure to the sun or UV light. While personal or self sunless tanning products are the most popular method of obtaining a sunless tan because they are inexpensive and can be done in privacy, the results can be poor because the application of the sunless tanning composition is often messy and difficult. Furthermore, methods of applying sunless tanning composition that utilize professionals or machinery such as spray booths are expensive and are subject to the skill of the professional or the machine functioning properly. Additionally, these methods can be embarrassing or uncomfortable to people because they require the user to disrobe in a public place and in some cases in front of others.

Methods of applying self sunless tanning products that are well known in the art include sunless tanning compositions that are applied by hand and square towelettes that are pre-soaked with the sunless tanning composition and are designed to be rubbed against the skin. A disadvantage of these prior art applications is that they require the user's hands to come into contact with the sunless tanning composition. Consequently, the active ingredients of the sunless tanning composition are frequently left on the palms or the heels of the hands, between fingers, and under the fingernails of the user.

Additionally, prior art methods for application of sunless tanning compositions require washing of the hands after application. Washing and/or rinsing of the hands immediately after the application of sunless tanning composition causes additional negative side-effects including streaking on the tops of hands where water has rinsed, and often rinsing will not prove completely effective in preventing undesired discoloration of the palms and fingers. Furthermore, with prior art applications it is frequently very difficult to apply the sunless tanning composition evenly and often streaks are left on the skin.

Accordingly, it is an object of the present invention to provide a sunless tanning system that provides for improved cleanliness and efficiency in application by preventing the user's hands from making contact with the sunless tanning composition.

It is another object of the present invention to provide a sunless tanning system that provides a more accurate and efficient application of a sunless tanning composition by providing a more uniform application of the sunless tanning composition while preventing the user's hands from being stained through contact with the sunless tanning composition.

Yet another object of the present invention is to provide an applicator with a pre-measured quantity of sunless tanning composition that is easy to use and conveniently packaged to facilitate an efficient application of sunless tanning composition by an individual on oneself.

### SUMMARY OF THE INVENTION

The foregoing objects are met by the present invention directed to an improved sunless tanning system. The sunless tanning system features a sunless tanning applicator in the shape of a mitt for the hand. The mitt has an interior to allow the hand to fit inside and an exterior for application of the sunless tanning composition. The mitt includes an insertion region at one end to allow the user's hands to extend into the mitt's interior. The sunless tanning composition is pre-impregnated into the exterior of the mitt thereby eliminating the need for the user to apply the sunless tanning composition to the applicator.

The surface of the mitt may be even and smooth in order to provide a uniform application of the sunless tanning composition. The self-tanning composition may additionally contain skin conditioners, emollients and other such ancillary ingredients and formulations.

The mitt is constructed to prevent the sunless tanning composition from penetrating into the mitt's interior. The mitt is packaged in a leak-proof, airtight encasement for hygiene, sterility, and stability. The packaging of the mitt is constructed to provide or allow the user to create an aperture in an end of the packaging and an aperture in the insertion region of the mitt without touching the mitt. An end of the packaging is generally aligned with the insertion region of the mitt so that the formation of the aperture in the packaging end allows access to the aperture in the insertion region of the mitt. This construction allows the user to insert his hand directly into the mitt without making contact with the outer surface of the mitt or any other part of the mitt or packaging which may contain the sunless tanning composition. Once a user opens the packaging and inserts a hand into the mitt, the user can easily withdraw the worn mitt from the packaging and then use the applicator to apply the sunless tanning composition on their body.

The sunless tanning system comprising the disclosed mitt and packaging therefor allows the user's hand to remains dry prior, during, and after application never coming in contact with the sunless tanning composition during application.

### DESCRIPTION OF THE DRAWINGS

The above and other objects, features and advantages of the present invention will become apparent from the following description of a preferred but, nonetheless, illustrative embodiment of the invention, taken in conjunction with the accompanying drawings, wherein

FIG. 1 provides a top plan view of the mitt-shaped applicator.

FIG. 2 provides a cross sectional view of the mitt-shaped applicator.

FIG. 3 provides a top plan view of the packaging with the mitt-shaped applicator enclosed therein depicted in phantom.

FIG. 4 is a top perspective view illustrating the mitt-shaped applicator inside the opened packaging.

FIG. 5 is a top perspective view illustrating the mitt-shaped applicator inside the opened packaging with the packaging manipulated to form an aperture in the insertion region of the mitt and an aperture in the packaging end.

FIG. 6 is a top perspective view illustrating an alternative embodiment of the packaging wherein a spring wire is placed in the packaging access end and an adhesive substance secures the mitt panels to the packaging panels creating access to the mitt's interior when the packaging is opened.

### DETAILED DESCRIPTION OF THE PREFERRED EMBODIMENTS

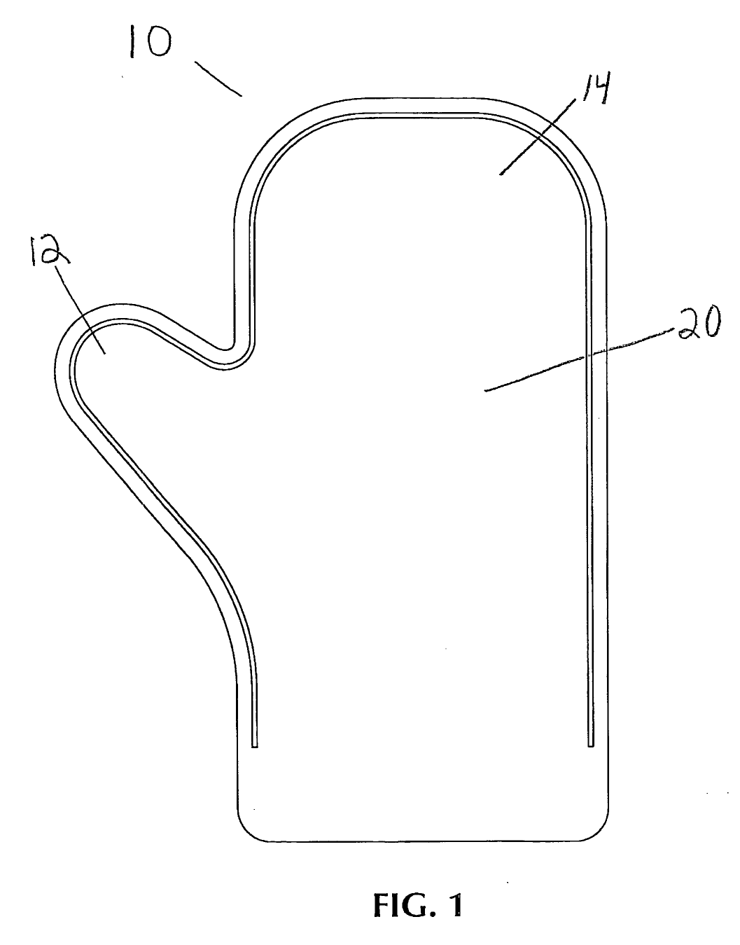

With particular reference to the drawings, the present invention is directed to an improved sunless tanning system comprising a sunless tanning applicator and packaging therefor. FIG. 1 illustrates a preferred embodiment of the sunless tanning applicator wherein the shape of the applicator 10 is of a mitt or “mitten” for the hand. In other embodiments, the applicator mitt may be configured without a separate single digit segment for the thumb 12. The applicator 10 can also be in the shape of a glove or can have another shape that allows the user's hand to fit inside and apply the sunless tanning composition.

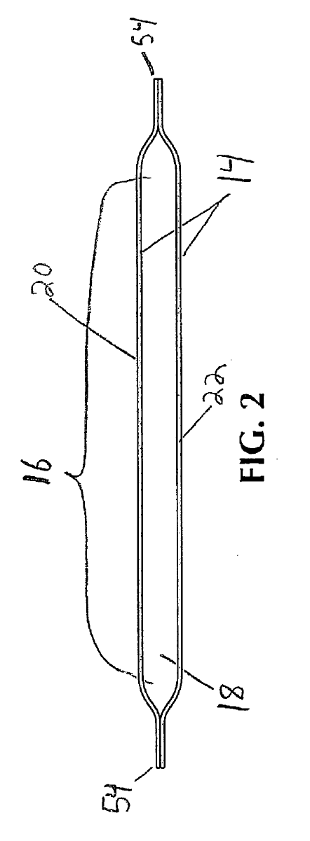

FIG. 2 illustrates a cross-sectional view of the applicator mitt. The mitt may be constructed of two panels 14 of material bonded to create a seal 54 along the periphery and forming a mitt-like construction. One end of the mitt is left unbonded to create an insertion region 16 that provides hand access into the mitt's interior 18.

The sunless tanning composition is pre-impregnated into one or both sides of the mitt's exterior 20. The exterior of the mitt 20 contains an absorbent “cosmetic-wipe” type material such as “Sontara EC Smooth” material. The absorbent material on the exterior of the mitt 20 is capable of sufficiently being impregnated with a sunless tanning composition typically, but not limited to comprising Dihydroxyacetone (DHA) as the key active ingredient. The exterior can additionally contain skin conditioners moisturizers, emollients, and other ingredients.

The surface of the mitt's exterior 20 has a smooth finish in order to provide a uniform application of the sunless tanning composition. Alternatively, the exterior surface 20 can contain an appreciable amount of wood pulp fibers or like materials so as to provide exfoliation of dead skin during application of the sunless tanning composition. Thus a cleansing effect as well as a tanning effect is obtained.

The mitt 10 is constructed to prevent the sunless tanning composition from penetrating into the mitt's interior 18. In a preferred embodiment, the interior of the mitt 18 has a barrier 22 preventing the composition impregnated on the exterior 20 from penetrating through the mitt and contacting the wearer's hand. As an example, the barrier 22 can be made of a layer made of a hydrophobic substance placed between the mitt's exterior 20 and the mitt's interior 18.

The packaging for the mitt 24 includes a leak-proof, airtight encasement 26 for hygiene, sterility, and stability. The packaging 24 may be made, for example, of aluminum foil, silver polyester laminate, or polyester laminate with a foil lining.

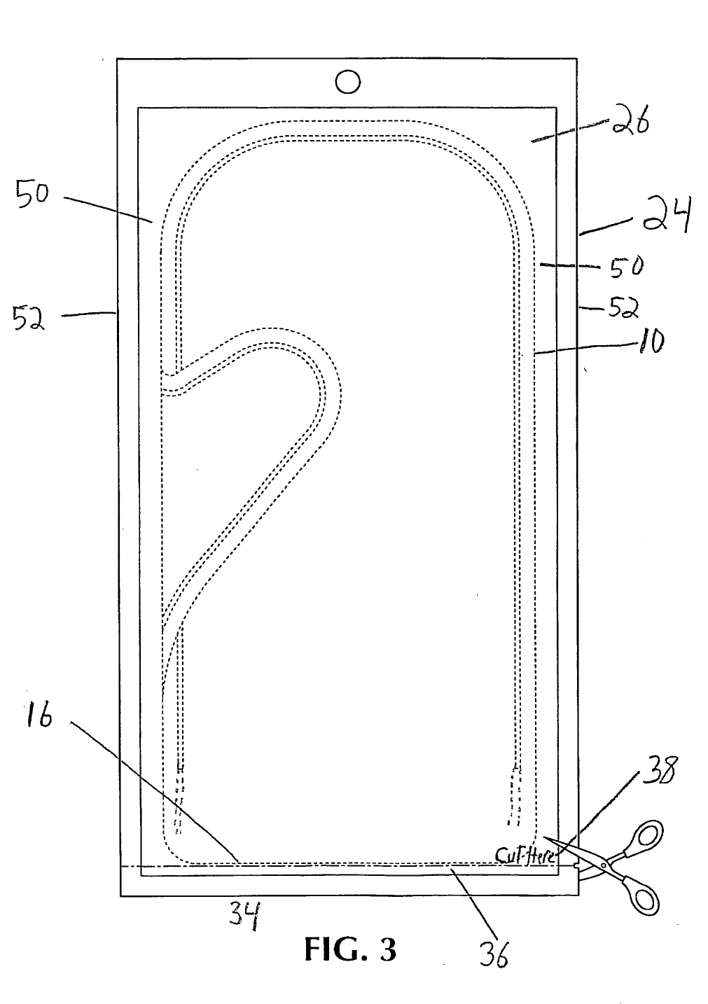

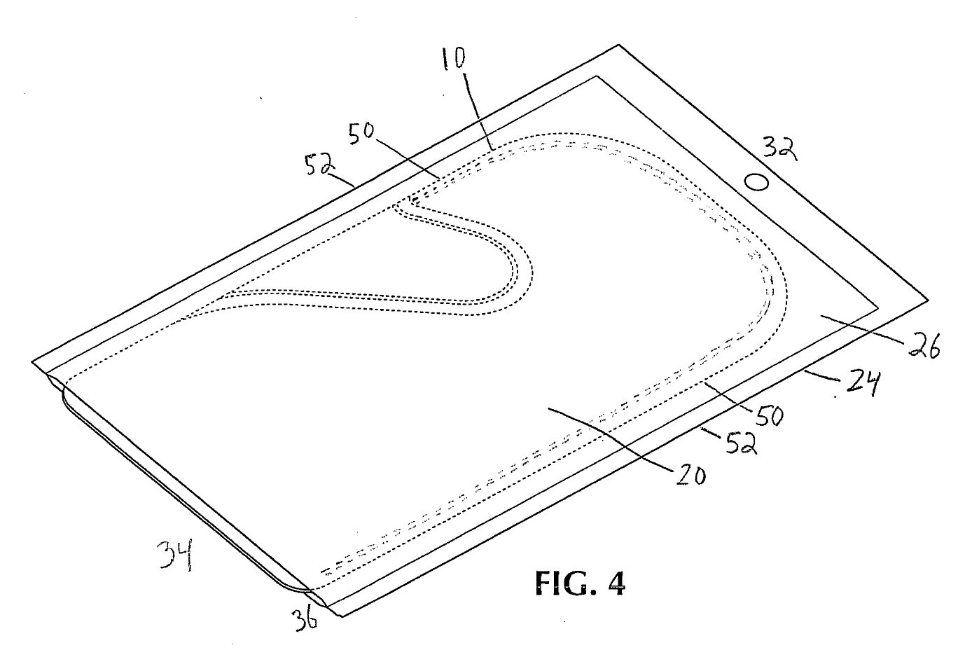

As referenced in FIGS. 4-6, the packaging of the mitt 24 is constructed with a top panel 28, bottom panel 30, a sealed end 32, and an access end 34 that is intended to be cut or torn by the user to break the encasement 26 of the packaging and allow the user to access the mitt 10. In a preferred embodiment as illustrated in FIG. 3, the packaging 24 is clearly marked with instructions 38 for the user to cut the packaging at an entry area 36 on the packaging's access end 34 just past the insertion region 16 of the mitt 10 lying inside the packaging. In another embodiment, an entry area 36 on the packaging's access end 34 just past the mitt's insertion region 16 is manufactured to be easily torn by the user and the packaging is marked with instructions therefor. The top panel 28 and bottom panel 30 are sealed together about their collective periphery with an appropriate adhesive or the like as known in the art to provide a leak-proof enclosure for the mitt.

The packaging of the mitt 24 and the orientation of the mitt 10 lying inside the packaging provide the user with access to the protected interior of the mitt 18 without coming into contact with the impregnated exterior 20 of the mitt or any part of the interior of the packaging 44 which may contain residual sunless tanning composition from the mitt exterior. The worn mitt 10 can then be withdrawn from the packaging 24 on the user's hand.

In the embodiment illustrated in FIGS. 3-6, the access forming means includes the packaging configured so that the top packaging panel 28 and bottom packaging panel 30 closely overlie the mitt panels 14. The mitt's discrete thumb segment 12 can be folded over the main body portion of the mitt 10 so that the side edges of the mitt 50 are clearly aligned to the side edges of the packaging 52.

The mitt 10 is aligned with the packaging 24 with an orientation such that the mitt's insertion region 16 is generally aligned with the packaging's access end 34. The packaging of the mitt 24 is constructed to allow the user to form a hand-access aperture 40 in the insertion region of the mitt 16 and an overlying aperture 42 in the access end 34 of the packaging without contacting the mitt. The packaging's access end aperture 42 when formed completely surrounds the mitt's insertion region aperture 40 to allow access to the entirety of the mitt's interior 18.

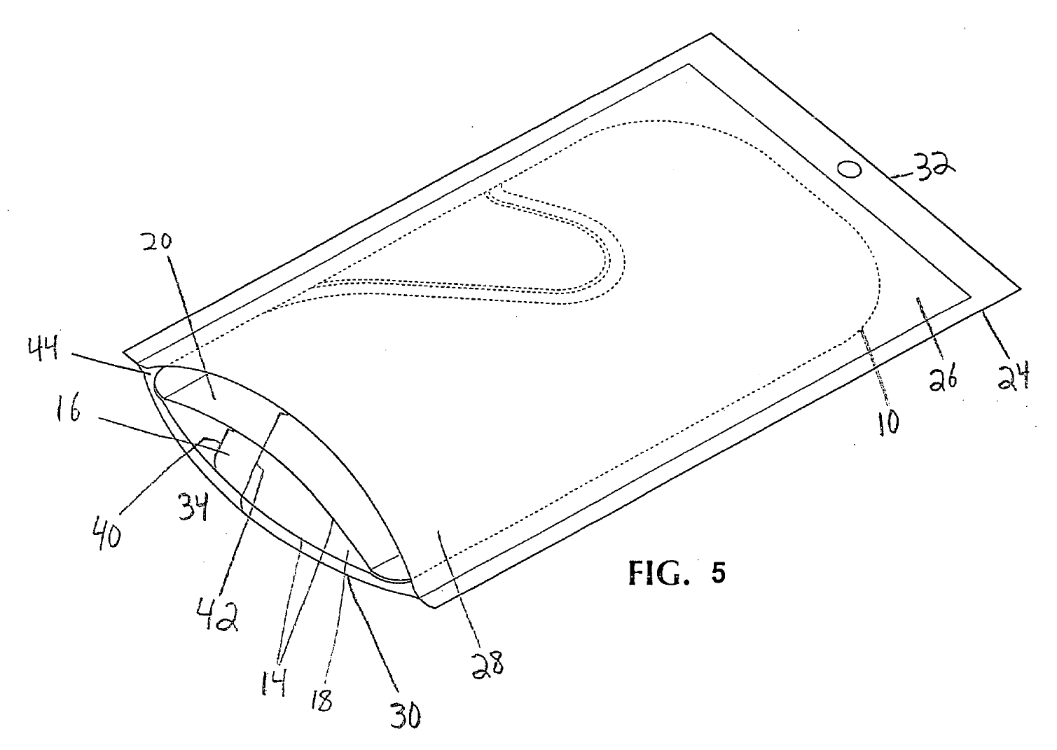

As illustrated in FIG. 5, the closely overlying orientation of the packaging panels 28, 30 with respect to the mitt panels 14 allows the user to engage the mitt panels 14 through and with the packaging panels 30 and simultaneously pull the opposing mitt panels 14 and corresponding packaging panels 30 apart to open the packaging access end aperture 42 and mitt insertion region aperture 40 thereby providing access to the mitt's interior 18. The mitt insertion region aperture 40 and the packaging access end aperture 42 are large enough to allow the user to easily put a hand directly inside the protected interior of the mitt 18 without coming into contact with the impregnated exterior 20 of the mitt or any part of the interior of the packaging 44 which may contain residual sunless tanning composition.

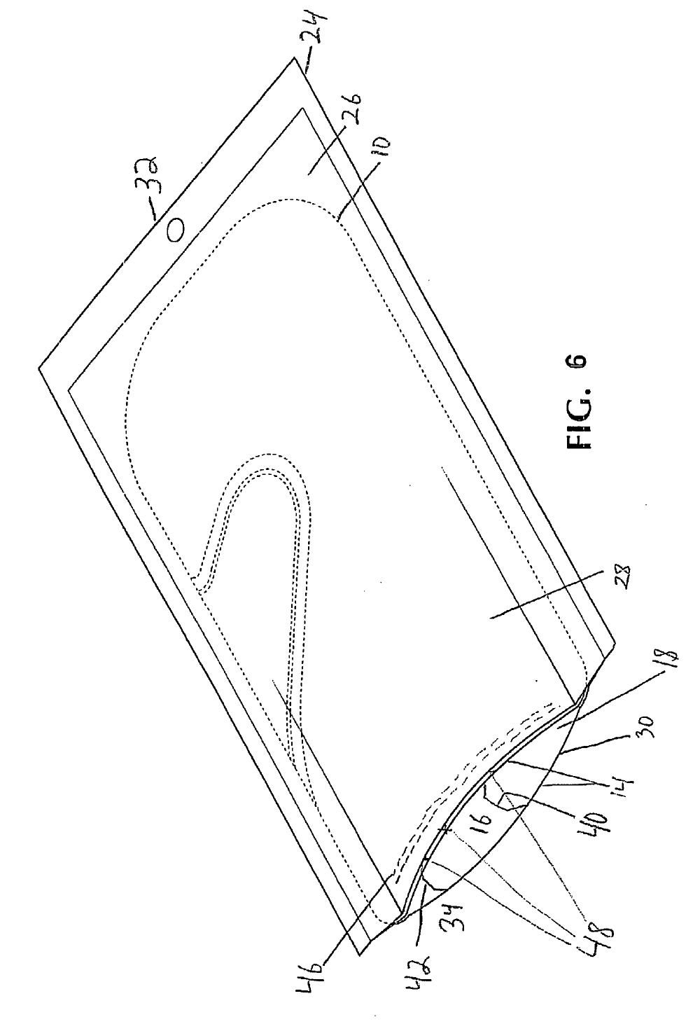

In another embodiment of the closely overlying packaging, there is an adhesive substance that secures one or more panels of the mitt 14 to the adjacent inner surface of the top packaging panel 28 or bottom packaging panel 30. It is preferable that the adhesive substance be weak so that the bond between the panels of the mitt 14 and the adjacent packaging panel can be readily broken by the user without damaging the mitt 10. The weak adhesive substance can be placed in any general region near the aperture between the panels of the mitt 14 and the adjacent packaging panel. As illustrated in FIG. 6, the adhesive substance is located at several points 48 between the mitt's panel 14 near the insertion region 16 and the packaging panels 28, 30 near the packaging access end 34. This construction allows the user to pull the top packaging panel 28 and bottom packaging panel 30 apart from each other to form a mitt insertion region aperture 40 and a packaging access end aperture 42 without requiring the user to specifically engage the mitt panels 14 through the packaging panels 28, 30.

In all embodiments where the user is required to manipulate the top packaging panel 28 or bottom packaging panel 30 in order to form a packaging access end aperture 42 and a mitt insertion region aperture 40 it is preferable that the packaging panels be constructed of a material that is supple to easily permit movement of the packaging panels.

In a further embodiment as illustrated in FIG. 6, a spring wire 46 or the like is attached to the top packaging panel 28 and/or on the bottom packaging panel 30 in an area just above the entry area 36 on the packaging's access end 34. Alternatively, the spring wire 46 can be attached to one or more mitt panels 14. The spring wire 46 applies a force that bends the packaging panel it is in contact with in a direction away from the opposing packaging panel. The force of the spring wire is opposed by the integrity of the sealed packaging on the packaging's access end. When the packaging's access end 34 is tom or cut by the user the integrity of the packaging in this area is broken and the spring wire 46 flexes to bend the packaging panel(s) it extends across in a direction away from the opposing packaging panel. This construction creates an aperture in the packaging's access end 34 immediately upon the user's opening of the packaging.

To further enhance the usefulness of this embodiment, it is preferable that an adhesive substance is used to secure one or more panels of the mitt 14 to the adjacent top packaging panel 28 or bottom packaging panel 30. Consequently, once the packaging is opened, a mitt insertion region aperture 40 and a packaging access end aperture 42 that extends beyond the insertion region aperture 40 will be formed thereby providing access to the mitt's interior 18 without requiring the user to perform any adjustments to the packaging 24.

The mitt 10 is oriented inside its packaging 24 with a flatly lying orientation which keeps the mitt panels 28, 30 at the insertion region 16 together. This prevents the sunless tanning composition from penetrating the mitt's interior 18 through the insertion region 16 while the glove is in its packaging 24. In another embodiment, the mitt panels may be fastened together by sealing means 56 placed on the inside surface of the mitt panels 28, 30 at the insertion region 16. The sealing means may be a weak adhesive, velcro, a zip-lock type fastener, or any other method well known in the art. The sealing means is designed to be easily broken without requiring any additional manipulation by the user. The sealing means is designed to break when the packaging is opened at the entry area 36 or when the mitt insertion region aperture 40 is formed. In one embodiment, the sealment means extends past the entry area and closely underlies the packaging 24 so that when the packaging is tom or cut the sealment means are broken and the mitt panels 28, 30 at the insertion region 16 are unattached.

What is claimed is:

1. A sunless tanning system comprising:
an applicator to apply sunless tanning composition wherein said applicator includes an applicator body having a top panel and bottom panel and an opening into an applicator interior, said applicator having an exterior impregnated with a sunless tanning composition, said applicator opening and applicator interior having dimensions to allow a hand to fit into said applicator interior, said applicator panels containing interior protecting means to prevent the sunless tanning composition from penetrating into said applicator interior; and
packaging for said applicator wherein said applicator packaging includes a top packaging panel and bottom packaging panel, an entry area where said packaging is opened by the user, and access means to afford the user access to the applicator interior without coming into contact with the applicator exterior or any part of the interior of the packaging which may contain the sunless tanning composition.

2. The sunless tanning system as set forth in claim 1 wherein said applicator is in the shape of a mitten.

3. The sunless tanning system as set forth in claim 1 wherein the exterior of the applicator is even and smooth.

4. The sunless tanning system as set forth in claim 1 wherein said applicator exterior contains an exfoliating composition.

5. The sunless tanning system as set forth in claim 4 wherein said exfoliating composition is wood pulp fibers.

6. The sunless tanning system as set forth in claim 1 wherein the sunless tanning composition is Dihydroxyacetone.

7. The sunless tanning system as set forth in claim 1 wherein said applicator exterior also contains skin conditioners.

8. The sunless tanning system as set forth in claim 1 wherein the interior protecting means consists of a hydrophobic barrier surrounding said applicator interior.

9. The sunless tanning system as set forth in claim 1 wherein said packaging is airtight and leak proof when unopened.

10. The sunless tanning system as set forth in claim 9 wherein said packaging includes a foil lining.

11. The sunless tanning system as set forth in claim 1 wherein said access means comprises said applicator opening aligned with said packaging entry area; said packaging panels being constructed of a supple material to permit movement of said packaging panels; said packaging panels having an orientation that closely overlies said applicator panels allowing the user to engage said applicator panels through said packaging and simultaneously spread said packaging panels and said applicator panels apart to form an applicator opening aperture and a packaging entry area aperture that extends beyond the applicator opening aperture to provide access to the applicator interior.

12. The sunless tanning system as set forth in claim 1 wherein said access means includes said applicator opening aligned with said packaging entry area and attaching means to affix at least one of said applicator panels to said packaging panels; said packaging panels being constructed of a supple material to permit movement of said packaging panels allowing the user to spread apart said packaging panels and form an applicator opening aperture and a packaging entry area aperture that extends beyond the applicator opening aperture to provide access to the applicator interior.

13. The sunless tanning system as set forth in claim 12 wherein said attaching means comprises an adhesive composition.

14. The sunless tanning system as set forth in claim 1 wherein said access mean means comprises a bending member attached to at least one of the packaging panels with said bending member applying a force when the packaging is opened that bends the attached packaging panel in a direction away from the opposing packaging panel thereby forming an aperture and attaching means to affix at least one of said applicator panels to said packaging panels.

15. The sunless tanning system as set forth in claim 14 wherein said bending member comprises a spring wire.

16. The sunless tanning system as set forth in claim 14 wherein said attaching means comprises an adhesive composition.

17. The sunless tanning system as set forth in claim 1 wherein said access means comprises a bending member attached to at least one of said applicator panels with said bending member applying a force when the packaging is opened that bends the attached applicator panel in a direction away from the opposing applicator panel thereby forming an aperture in the applicator opening and an aperture in the packaging entry area to provide access to the applicator interior.

18. The sunless tanning system as set forth in claim 17 wherein said bending member comprises a spring wire.
